(19) United States
(12) Patent Application Publication
(10) Pub. No.: US 2024/0382419 A1
(43) Pub. Date: Nov. 21, 2024

(54) METERED DOSE INHALERS AND SOLUTION COMPOSITIONS

(71) Applicant: KINDEVA DRUG DELIVERY L.P., Woodbury, MN (US)

(72) Inventors: Philip COCKS, West Bridgford (GB); Alexander SLOWEY, Stapleford (GB); Benjamin MYATT, Leicester (GB); Sarah WRIGGLESWORTH, Loughborough (GB); James LISTER, Nottinghamshire (GB); Lauren HARRISON, Loughborough (GB); Chad HARALDSON, Apple Valley, MN (US); Joseph MILLER, Oakdale, MN (US); James DRAKE, Hudson, WI (US); Stephen STEIN, Lino Lakes, MN (US)

(21) Appl. No.: 18/689,555

(22) PCT Filed: Sep. 8, 2022
(86) PCT No.: PCT/US2022/042956

§ 371 Date: Mar. 6, 2024

Related U.S. Application Data
(60) Provisional application No. 63/241,677, filed on Sep. 8, 2021.
(60) Provisional application No. 63/315,337, filed on Mar. 1, 2022.
(60) Provisional application No. 63/328,120, filed on Apr. 6, 2022.

(51) Int. Cl. A61K 9/00; A61K 31/137; A61K 31/55; A61K 31/573; A61K 47/06; A61K 47/10; A61K 47/12; A61M 15/00

(52) U.S. Cl. CPC A61K 9/008; A61K 31/137; A61K 31/55; A61K 31/573; A61K 47/06; A61K 47/10; A61K 47/12; A61M 15/009

(57) ABSTRACT

A metered dose inhaler comprising a metering valve, a canister, an actuator comprising an actuator nozzle, wherein the canister comprises a formulation, the formulation comprising greater than 70% by weight of propellant HFO-1234ze(E), and at least one active pharmaceutical ingredient dissolved in the formulation to form a solution, said active pharmaceutical ingredient preferably selected from beclomethasone, formoterol or tiotropium.

The present application claims priority to U.S. Provisional Application No. 63/241,677, filed Sep. 8, 2021, U.S. Provisional Application No. 63/315,337,filed Mar. 1, 2022, and U.S. Provisional Application Number 63/328, 120, filed Apr. 6, 2022, all of which are incorporated herein by reference in their entirety.

### BACKGROUND

Delivery of aerosolized medicament to the respiratory tract for the treatment of respiratory and other diseases can be done using, by way of example, pressurized metered dose inhalers (pMDI), dry powder inhalers (DPI), or nebulizers. pMDIs are familiar to many patients who suffer from asthma or chronic obstructive pulmonary disease (COPD). pMDI devices can include an aluminum canister, sealed with a metering valve, that contains medicament formulation. Generally, a typical current medicament formulation includes one or more medicinal compounds present in a liquefied hydrofluoroalkane (HFA) propellant.

Historically, the propellants in most pMDIs have been chlorofluorocarbons (CFCs). However, environmental concerns during the 1990s led to the replacement of CFCs with hydrofluoroalkanes (HFAs) as the most commonly used propellant in pMDIs. Although HFAs do not cause ozone depletion, they do have a stated high global warming potential (GWP), which is a measurement of the future radiative effect of an emission of a substance relative to that of the same amount of carbon dioxide (CO₂). The two HFA propellants most commonly used in pMDIs are HFA-134a (CF₃CH₂F) and HFA-227 (CF₃CHFCHF₃) having stated 100-year GWP values of 1300 to 1430 and 3220 to 3350, respectively.

Various other propellants have been proposed over the years. Among them, hydrofluoroolefins (HFOs) and carbon dioxide (CO₂) have been mentioned as a potential propellant for pMDIs, but no pMDI product has been successfully developed or commercialized using either as a propellant.

### SUMMARY

It has now been found that despite HFO-1234ze(E)'s differences from other pMDI propellants, a practical pMDI can be made using HFO-1234ze(E). One advantage of such pMDIs is HFO-1234ze(E)'s stated GWP of less than 1.

In one embodiment, a pMDI (also referred to herein as an MDI or metered dose inhaler) is provided that includes: a metering valve; a canister; and an actuator that includes an actuator nozzle; wherein the canister includes a formulation (i.e., composition), the formulation including greater than 70% by weight of propellant HFO-1234ze(E), and at least one active pharmaceutical ingredient (API) dissolved in the formulation to form a solution. In certain embodiments, the formulation further includes ethanol. In certain embodiments, the API is selected from beta agonists (short- or long-acting beta agonists), corticosteroids, anticholinergic agents, tyrosine kinase (TYK) inhibitors, and combinations thereof.

In one embodiment, a metered dose inhaler is provided that includes: a metering valve; a canister; and an actuator that includes an actuator nozzle; wherein the canister includes a formulation, the formulation including a propellant including HFO-1234ze(E), and an active pharmaceutical ingredient including beclomethasone or a pharmaceutically acceptable salt or ester thereof (e.g., beclomethasone dipropionate), wherein the beclomethasone or pharmaceutically acceptable salt or ester thereof is dissolved in the formulation to form a solution.

In one embodiment, a metered dose inhaler is provided that includes: a metering valve; a canister; and an actuator that includes an actuator nozzle; wherein the canister includes a formulation, the formulation including a propellant including HFO-1234ze(E), and an active pharmaceutical ingredient including formoterol or a pharmaceutically acceptable salt or ester thereof (e.g., formoterol fumarate), wherein the formoterol or pharmaceutically acceptable salt or ester thereof is dissolved in the formulation to form a solution.

In one embodiment, a metered dose inhaler is provided that includes: a metering valve; a canister; and an actuator that includes an actuator nozzle; wherein the canister includes a formulation, the formulation including a propellant including HFO-1234ze(E), and an active pharmaceutical ingredient including formoterol or a pharmaceutically acceptable salt or ester thereof, and beclomethasone or a pharmaceutically acceptable salt or ester thereof, wherein the formoterol or pharmaceutically acceptable salt or ester thereof and the beclomethasone or pharmaceutically acceptable salt or ester thereof is dissolved in the formulation to form a solution.

In one embodiment, a metered dose inhaler is provided that includes: a metering valve; a canister; and an actuator that includes an actuator nozzle; wherein the canister includes a formulation, the formulation including a propellant including HFO-1234ze(E), and an active pharmaceutical ingredient including tiotropium or a pharmaceutically acceptable salt or ester thereof, wherein the tiotropium or pharmaceutically acceptable salt or ester thereof is dissolved in the formulation to form a solution.

Herein, “dissolved in the formulation” or “dissolved in the composition” means that the recited components (e.g., APIs) are dissolved in the propellant, or dissolved in the propellant and other components such as a cosolvent, to form a solution.

Herein, the term “comprises” and variations thereof do not have a limiting meaning where these terms appear in the description and claims. Such terms will be understood to imply the inclusion of a stated step or element, or group of steps or elements, but not the exclusion of any other step or element, or group of steps or elements. The phrase “consisting of” means including, and limited to, whatever follows the phrase “consisting of.” Thus, the phrase “consisting of” indicates that the listed elements are required or mandatory, and that no other elements may be present. The phrase “consisting essentially of” means including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may, or may not, be present depending upon whether or not they materially affect the activity or action of the listed elements. Any of the elements or combinations of elements that are recited in this specification in open-ended language (e.g., comprise and derivatives thereof), are considered to additionally be recited in closed-ended language (e.g., consist and derivatives thereof) and in partially closed-ended language (e.g., consist essentially and derivatives thereof).

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

Throughout this disclosure, singular forms such as “a,” “an,” and “the” are often used for convenience; singular forms are meant to include the plural unless the singular alone is explicitly specified or is clearly indicated by the context.

As used herein, the term “or” is generally employed in its usual sense including “and/or” unless the content clearly dictates otherwise.

The term “and/or” means one or all of the listed elements or a combination of any two or more of the listed elements.

The phrase “ambient conditions” as used herein, refers to an environment of room temperature (approximately 20° C. to 25° C.) and 30-60% relative humidity.

Also herein, all numbers are assumed to be modified by the term “about” and in certain embodiments, preferably, by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used. Herein, “up to” a number (e.g., up to 50) includes the number (e.g., 50). Herein, “at least” a number (e.g., at least 50) includes the number (e.g., 50). Herein, “no more than” a number (e.g., no more than 50) includes the number (e.g., 50).

Numerical ranges, for example “between x and y” or “from x to y”, include the endpoint values of x and y. Also herein, the recitations of numerical ranges by endpoints include all numbers subsumed within that range as well as the endpoints (e.g., 1 to 5 includes 1, 1.5, 2, 2.75, 3, 3.80, 4, 5, etc.).

Some terms used in this application have special meanings, as defined herein. All other terms will be known to the skilled artisan and are to be afforded the meaning that a person of skill in the art at the time of the invention would have given them.

Elements in this specification that are referred to as “common,” “commonly used,” “conventional,” “typical,” “typically,” and the like, should be understood to be common within the context of the compositions, articles, such as inhalers and pMDIs, and methods of this disclosure; this terminology is not used to mean that these features are present, much less common, in the prior art. Unless otherwise specified, only the Background section of this Application refers to the prior art.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

The present disclosure will be described with respect to embodiments and with reference to certain drawings, but the invention is not limited thereto. The drawings described are only schematic and are non-limiting. In the drawings, the size of some of the elements can be exaggerated and not drawn to scale for illustrative purposes.

The above summary of the present disclosure is not intended to describe each disclosed embodiment or every implementation of the present disclosure. The description that follows more particularly exemplifies illustrative embodiments. In several places throughout the disclosure, guidance is provided through lists of examples, which examples may be used in various combinations. In each instance, the recited list serves only as a representative group and should not be interpreted as an exclusive or exhaustive list. Thus, the scope of the present disclosure should not be limited to the specific illustrative structures described herein, but rather extends at least to the structures described by the language of the claims, and the equivalents of those structures. Any of the elements that are positively recited in this specification as alternatives may be explicitly included in the claims or excluded from the claims, in any combination as desired. Although various theories and possible mechanisms may have been discussed herein, in no event should such discussions serve to limit the claimable subject matter.

### BRIEF DESCRIPTION OF THE DRAWINGS

The present disclosure will be described with respect to embodiments and with reference to certain drawings, but the invention is not limited thereto. The drawings described are only schematic and are non-limiting. In the drawings, the size of some of the elements can be exaggerated and not drawn to scale for illustrative purposes.

FIG. 1 is a cross-sectional side view of an inhaler including a canister containing a valve according to the present disclosure.

FIG. 2 is a detailed cross-sectional side view of the inhaler of FIG. 1.

FIG. 3 is a cross-sectional side view of a metering valve for an inhaler.

### DETAILED DESCRIPTION

The formulations of the present disclosure are solutions (i.e., solution formulations or solution compositions). That is, the formulations include one or more APIs dissolved in the formulations (i.e., solubilized in the propellant and often a cosolvent and/or other components) to form solutions. Herein, a “solution” is a homogeneous solution that does not have particulate material visible to the unaided human eye.

Solution and suspension formulations are fundamentally different pMDI formulation approaches. Different factors need to be considered when undertaking the development of products using either of these formulation approaches. Accordingly, it is not possible to apply the same knowledge and understanding of suspension formulations to solution formulations. In solutions, solubility of the API in the propellant, and optional cosolvent, is the key consideration. Various strategies can be used to improve solubility via use of additional excipients such as polyethylene glycol or water. Typically, solutions give smaller aerosol particle size distributions than suspensions and are generally more efficient than suspensions, but the overall dose may be limited due to the amount of API that can be solubilized. The use of cosolvents in solution pMDIs influences droplet evaporation rates and can also lead to changes in the resulting solid-state particles formed in the lung, which could impact on pharmacological uptake of the API compared with deposited API from a suspension. Also, some APIs are at higher risk of chemical degradation in solution formulations and often require specific formulation strategies, such as the use of stabilizing acids and specific selection of container closure systems to maximize chemical stability. These problems are specific to solutions and any teachings specific to suspensions do not necessarily overcome them.

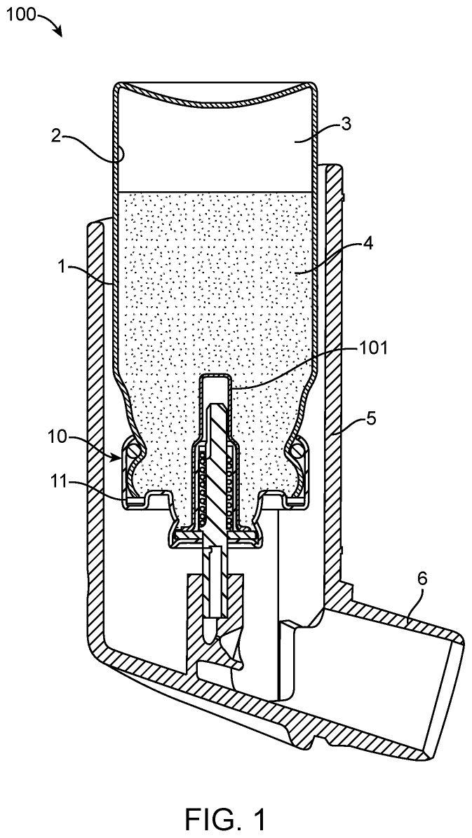

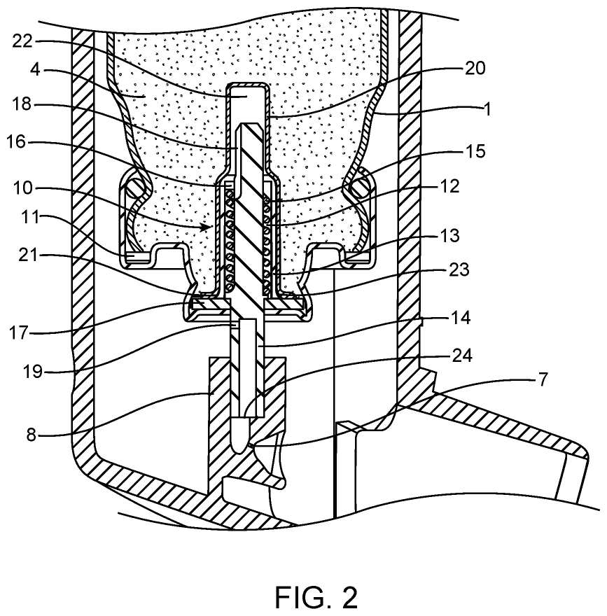

The various embodiments of formulations described herein can be utilized with any suitable inhaler. For example, FIG. 1 shows one embodiment of a metered dose inhaler 100, including an aerosol canister 1 fitted with a metered dose metering valve 10 (shown in its resting position). The metering valve 10 is typically affixed, i.e., crimped, onto the canister 1 via a cap or ferrule 11 (typically made of aluminum or an aluminum alloy) which is generally provided as part of the valve assembly. Between the canister and the ferrule there may be one or more seals. In the embodiments shown in FIGS. 1 and 2 between the canister 1 and the ferrule 11 there are two seals including, e.g., an O-ring seal and a gasket seal.

As shown in FIG. 1, the canister/valve dispenser is typically provided with an actuator 5 including an appropriate patient port 6, such as a mouthpiece. For administration to the nasal cavities the patient port is generally provided in an appropriate form (e.g., smaller diameter tube, often sloping upwardly) for delivery through the nose. Actuators are generally made of a plastic material, for example polypropylene or polyethylene. As can be seen from FIG. 1, inner walls 2 of the canister 1 and outer walls 101 of the portion(s) of the metering valve 10 located within the canister define a formulation chamber 3 in which aerosol formulation 4 is contained.

The valve 10 shown in FIGS. 1 and 2, includes a metering chamber 12, defined in part by an inner valve body 13, through which a valve stem 14 passes. The valve stem 14, which is biased outwardly by a compression spring 15, is in sliding sealing engagement with an inner tank seal 16 and an outer diaphragm seal 17. The valve 10 also includes a second valve body 20 in the form of a bottle emptier. The inner valve body 13 (also referred to as the “primary” valve body) defines in part the metering chamber 12. The second valve body 20 (also referred to as the “secondary” valve body) defines in part a pre-metering region or chamber besides serving as a bottle emptier.

Referring to FIG. 2, aerosol formulation 4 can pass from the formulation chamber 3 into a pre-metering chamber 22 provided between the secondary valve body 20 and the primary valve body 13 through an annular space 21 between a flange 23 of the secondary valve body 20 and the primary valve body 13. To actuate (fire) the valve 10, the valve stem 14 is pushed inwardly relative to the canister 1 from its resting position shown in FIGS. 1 and 2, allowing formulation to pass from the metering chamber 12 through a side hole 19 in the valve stem and through a stem outlet 24 to an actuator nozzle 7 then out to the patient. When the valve stem 14 is released, formulation enters into the valve 10, in particular into the pre-metering chamber 22, through the annular space 21 and thence from the pre-metering chamber through a groove 18 in the valve stem past the tank seal 16 into the metering chamber 12.

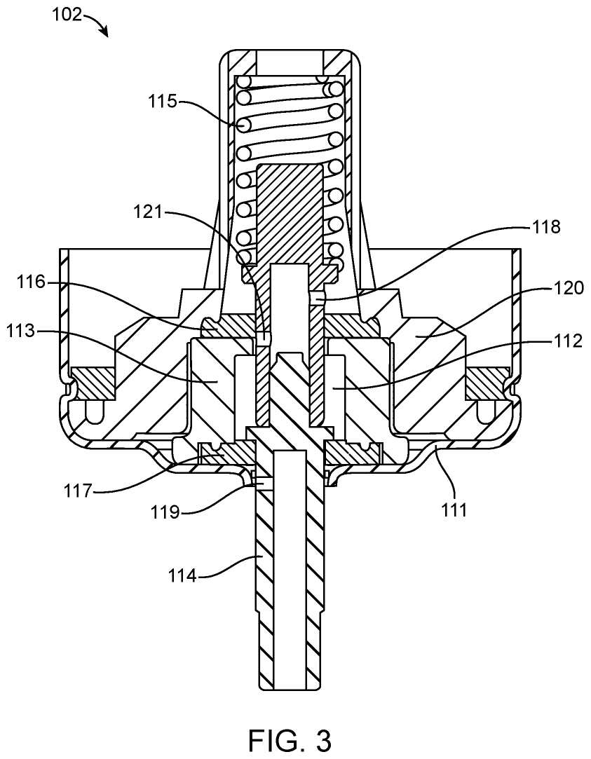

FIG. 3 shows another embodiment of a metered dose aerosol metering valve 102, different from the embodiment shown in FIGS. 1 and 2, in its rest position. The valve 102 has a metering chamber 112 defined in part by a metering tank 113 through which a stem 114 is biased outwardly by spring 115. The stem 114 is made in two parts that are push fit together before being assembled into the valve 102. The stem 114 has an inner seal 116 and an outer seal 117 disposed about it and forming sealing contact with the metering tank 113. A valve body 120 crimped into a ferrule 111 retains the aforementioned components in the valve. In use, formulation enters the metering chamber via orifices 121 and 118. The formulation's outward path from the metering chamber 112 when a dose is dispensed is via orifice 119.

The primary propellant of compositions (i.e., formulations) according to the disclosure is HFO-1234ze(E), also known as trans-1,1,1,3-tetrafluoropropene, trans-1,3,3,3-tetrafluoropropene, or trans-1,3,3.3-tetrafluoroprop-1-ene. The chemical structure of trans and cis isomers of HFO-1234ze are very different. As a result, these isomers have very different physical and thermodynamic properties. The significantly lower boiling point and higher vapor pressure of the trans (E) isomer relative to that of the cis (Z) isomer, at ambient conditions, makes the trans isomer a far more thermodynamically suitable propellant for achieving efficient pMDI atomization.

In some embodiments, the amount of HFO-1234ze(E) by weight in the composition is greater than 70%, at least 80%, greater than 80%, at least 85%, greater than 85%, at least 90%, or greater than 90%. In some embodiments, the amount of HFO-1234ze(E) by weight is between 80% and 99%, between 80% and 98%, between 80% and 95%, or between 85% and 90%. In some embodiments, HFO-1234ze(E) is essentially the sole propellant in the composition. That is, the pharmaceutical product performance parameters, such as emitted dose and emitted particle size distribution, are not significantly different than if HFO-1234ze(E) were the sole propellant in the composition. In some embodiments, the amount of HFO-1234ze(E) by weight of the total propellant in the composition is greater than 95%, greater than 98%, greater than 99%, greater than 99.5%, and greater than 99.8%.

The propellant HFO-1234ze(E) is very different from an alternative low GWP propellant HFA-152a. These two propellants have different physical, chemical, and thermodynamic properties such as boiling point, vapor pressure, water solubility, liquid density, surface tension, etc. The differences in these properties make replacing one propellant with another without significantly compromising or altering pMDI product performance difficult to achieve. For example, the thermodynamic differences in propellant boiling point and vapor pressure can significantly affect pMDI aerosolization efficiency and give rise to differences in primary and secondary atomization mechanisms. Differences in dipole moment and polarity between the propellants can affect the solubility of drugs and excipients in the formulation. Differences in hygroscopicity between the propellants can affect moisture uptake, which could be problematic for solution formulations, particularly if physical stability due to moisture uptake or chemical degradation in which water is involved is likely. Chemical interactions of the different propellants with drug and excipients may also be significantly different, which could affect the long-term chemical stability of the product over the intended shelf life. The two propellants interact chemically and physically with valve plastics and elastomeric components, which could give rise to differences in the types and amounts of extractables and leachables, as well as impacting mechanical valve function. The thermodynamic properties of the propellants can give rise to different droplet particle sizes due to different evaporation rates and can also result in differences in spray characteristics such as spray force, temperature, and spray duration. Historically, the transition from CFC to HFA propellants has required significant efforts to develop new approaches to reformulate and develop capable hardware to achieve appropriate pMDI product performance. That is, it was not possible to simply directly substitute one propellant for another. Changing between propellant HFA-152a to HFO-1234ze(E) in a pMDI is equally challenging due to many of the factors highlighted above.

In some embodiments, other propellants, such as hydrofluoroalkanes, including HFA-134a, HFA-227 (1,1,1,2,3,3,3-heptafluoropropane), or HFA-152a, may be included as a minor component. Still other propellants that may be included as a minor component include other hydrofluoroolefins, including HFO-1234yf (2,3,3,3-tetrafluoropropene) and HFO-1234ze(Z) (i.e., cis-HFO-1234ze). Thus, in some embodiments, the differences between HFA-152a and HFO-1234ze(E) discussed herein can be utilized to advantage by using a minor amount of HFA-152a. Amounts of such secondary propellants can include 0.1% to 20%, 0.1% to 5%, or 0.1% to 0.5%, by weight, of the total composition (i.e., total formulation).

The total amount of composition is desirably selected so that at least a portion of the propellant in the canister is present as a liquid after a predetermined number of medicinal doses have been delivered. The predetermined number of doses may be 5 to 200, 30 to 200, 60 to 200, 60 to 120, 60, 120, 200, or any other number of doses. The total amount of composition in the canister may be from 1.0 grams (g) to 30.0 g, 2.0 g to 20.0 g, or 5.0 to 10.0 g. The total amount of composition is typically selected to be greater than the product of the predetermined number of doses and the metering volume of the metering valve. In some embodiments, the total amount of composition is greater than 1.1 times, greater than 1.2 times, greater than 1.3 times, greater than 1.4 times, or greater than 1.5 times the product of the predetermined number of doses and the metering volume of the metering valve. This typically ensures that the amount of each dose remains relatively constant through the life of the inhaler.

The API may be a drug, vaccine, DNA fragment, hormone, other treatment, or a combination of any two or more APIs. In certain embodiments, the formulations may include at least two (in certain embodiments, two or three, and in certain embodiments, two) APIs in solution.

The API may be provided in any form suitable for formulation as a solution. In certain embodiments, the API may be provided as a solid, such as a powder or a micronized powder, or as a liquid, such as a stock solution. Any suitable form of API compatible with preparation of a solution may be used for the formulations of the present disclosure.

Exemplary APIs can include those for the treatment of respiratory disorders, e.g., a bronchodilator, such as a short- or long-acting beta agonist, an anti-inflammatory (e.g., a corticosteroid), an anti-allergic, an anti-asthmatic, an antihistamine, a TYK inhibitor, or an anticholinergic agent. Exemplary APIs can include terbutaline, ipratropium, oxitropium, tiotropium, beclomethasone, flunisolide, ciclesonide, cromolyn sodium, nedocromil sodium, ketotifen, azelastine, ergotamine, cyclosporine, aclidinium, umeclidinium, glycopyrronium (i.e., glycopyrrolate), salmeterol, formoterol, procaterol, indacaterol, carmoterol, milveterol, olodaterol, vilanterol, abediterol, omalizumab, zileuton, insulin, pentamidine, calcitonin, leuprolide, alpha-I-antitrypsin, interferon, triamcinolone, nintedanib, a pharmaceutically acceptable salt or ester of any of the listed drugs, or a mixture of any of the listed drugs, their pharmaceutically acceptable salts or their pharmaceutically acceptable esters. For beclomethasone, an exemplary ester is propionate.

In all embodiments, the API(s) are dissolved in the formulation (i.e., as a solution). In the event that a combination of two or more APIs are used, all of the APIs are in solution.

In one embodiment, the formulation has beclomethasone or a pharmaceutically acceptable salt or ester thereof as the sole API, more particularly beclomethasone dipropionate.

In one embodiment, the formulation has formoterol or a pharmaceutically acceptable salt or ester thereof as the sole API, more particularly formoterol fumarate.

In one embodiment, the formulation includes tiotropium or a pharmaceutically acceptable salt or ester thereof as the sole API, more particularly tiotropium bromide.

In one embodiment, the formulation includes beclomethasone and formoterol or pharmaceutically acceptable salts or esters thereof, more particularly beclomethasone dipropionate and formoterol fumarate, and more particularly where both active ingredients are dissolved in the formulation.

The amount of API may be determined by the required dose per actuation and the pMDI metering valve size, that is, the size of the metering chamber, which may be between 5 microliters (μL or mcl) and 200 microliters, between 25 microliters and 200 microliters, between 25 microliters and 150 microliters, between 25 microliters and 100 microliters, between 50 microliters and 100 microliters, between 25 microliters and 65 microliters, between 50 microliters and 65 microliters, or between 50 microliters and 63 microliters. The concentration of each API is typically from 0.0008% to 3.4% by weight, or 0.01% to 1.0% by weight, sometimes from 0.05% to 0.5% by weight, and as such, the medicament makes up a relatively small percentage of the total composition.

In certain embodiments, typical formulations of the present disclosure include the API in an amount of at least 0.001 milligram per actuation (mg/actuation), or at least 0.001 mg/actuation. In certain embodiments, typical formulations of the present disclosure include the API in an amount of less than 0.5 mg/actuation.

In embodiments, typical formulations of the present disclosure include the API in an amount of at least 1 μg/actuation, at least 10 μg/actuation, at least 50 μg/actuation, at least 100 μg/actuation, at least 150 μg/actuation, at least 200 μg/actuation, at least 300 μg/actuation, or at least 400 μg/actuation. In embodiments, typical formulations of the present disclosure include the API in an amount of less than 500 μg/actuation, at most 400 μg/actuation, at most 300 μg/actuation or at most 200 μg/actuation. In some preferred embodiments, formulations of the present disclosure include the API in an amount of 80 μg/actuation to 120 μg/actuation.

In some embodiments, additional components (e.g., excipients) beyond propellant and API can be added to the formulation. These components may have various uses and functions, including, but not limited to, aiding in dissolution of API or other components, and/or aiding in chemical stabilization of API or other components.

In some embodiments, a cosolvent is included. One particularly useful cosolvent is ethanol. In some embodiments, ethanol is used as a cosolvent in solution formulations, i.e., where the API is dissolved in the formulation. In one aspect, the ethanol may aid in dissolving the API whereas the API may not be soluble in the formulation in the absence of ethanol. When used in solution formulations, ethanol may be in amounts on a weight percent basis of the total formulation of at least 0.5%, at least 1%, at least 2%, at least 5%, at least 10%, at least 15%. When used in solution formulations, ethanol may be in amounts on a weight percent basis of the total formulation of up to 20% or up to 15%.

In certain embodiments, when used in solution formulations, ethanol may be in amounts on a weight percent basis of the total formulation of between 0.5% and 20%, between 1% and 20%, between 2% and 20%, between 2% and 15%, between 5% and 15%, between 10% and 15%, or between 15% and 20%. In some embodiments, the ethanol content is greater than 17%, or at least 17.5%, on a weight percent basis of the total formulation.

In some embodiments, an acid can also be used to facilitate dissolution and/or stabilization of an API in the formulation via modification of the hydrogen ion concentration in the formulation. However, acid-free formulations can be advantageous for some purposes, and acid is not required unless otherwise specified.

In embodiments wherein an acid is included, the acid may be an organic acid, inorganic acid, or a combination thereof. In certain embodiments, the acid is an inorganic acid. Exemplary inorganic acids include hydrochloric acid, nitric acid, phosphoric acid, sulfuric acid, and a combination thereof. In certain embodiments, the acid is an organic acid. Exemplary organic acids include citric acid, ascorbic acid, acetic acid, maleic acid, fumaric acid, succinic acid, formic acid, propionic acid, oxalic acid, lactic acid, glycolic acid, and a combination thereof. When used, the amount of acid on a weight percent basis of the total formulation is between 0.001% and 1.0%, between 0.002% and 0.5%, between 0.004% and 0.4%, or between 0.04% and 0.4%.

In some embodiments, additional excipients such as polyethylene glycol (e.g., PEG 300 or PEG 1000) or water may be used to enhance solubility. Typically, no more than 1% by weight (based on the total weight of the formulation) water or polyethylene glycol would be used as an excipient in a formulation of the present disclosure. Typically, at least 0.01% by weight (based on the total weight of the formulation) water or polyethylene glycol would be used as an excipient in a formulation of the present disclosure.

In certain embodiments, compositions of the present disclosure preferably display physical stability such that no particles are visible for at least 18 months, and often from 18 to 36 months under typical storage conditions (e.g., room temperature). In certain embodiments, compositions of the present disclosure preferably display chemical stability such that no degradation products are formed for at least 18 months, and often from 18 to 36 months under typical storage conditions (e.g., room temperature).

Returning to FIG. 1, in use, the patient actuates the inhaler 100 by pressing downwardly on the canister 1. This moves the canister 1 into the body of the actuator 5 and presses the valve stem 14 against the actuator stem socket 8 resulting in the canister metering valve 10 opening and releasing a metered dose of composition that passes through the actuator nozzle 7 and exits the mouthpiece 6 into the patient's mouth. It should be understood that other modes of actuation, such as breath-actuation, may be used as well and would operate as described with the exception that the force to depress the canister would be provided by the device, for instance by a spring or a motor-driven screw, in response to a triggering event, such as patient inhalation.

Devices that may be used with medicament compositions of the present disclosure include those described in U.S. Pat. No. 6,032,836 (Hiscocks et al.), U.S. Pat. No. 9,010,329 (Hansen), and U.K. Patent GB 2544128 B (Friel).

The metered dose inhaler can include a dose counter for counting the number of doses. Suitable dose counters are known in the art, and are described in, for example, U.S. Pat. No. 8,740,014 (Purkins et al.); U.S. Pat. No. 8,479,732 (Stuart et al.); and U.S. Pat. No. 8,814,035 (Stuart), and U.S. Patent Application Publication No. 2012/0234317 (Stuart), all of which are incorporated by reference in their entirety with respect to their disclosures of dose counters.

One exemplary dose counter, which is described in detail in U.S. Pat. No. 8,740,014 (Purkins et al., hereby incorporated by reference in its entirety for its disclosure of the dose counter) has a fixed ratchet element and a trigger element that is constructed and arranged to undergo reciprocal movement coordinated with the reciprocal movement between an actuation element in an inhaler and the dose counter. The reciprocal movement can include an outward stroke (outward being with respect to the inhaler) and a return stroke. The return stroke returns the trigger element to the position that it was in prior to the outward stroke. A counter element is also included in this type of dose counter. The counter element is constructed and arranged to undergo a predetermined counting movement each time a dose is dispensed. The counter element is biased towards the fixed ratchet and trigger elements and is capable of counting motion in a direction that is substantially orthogonal to the direction of the reciprocal movement of the trigger element.

The counter element in the above-described dose counter includes a first region for interacting with the trigger member. The first region includes at least one inclined surface that is engaged by the trigger member during the outward stroke of the trigger member. This engagement during the outward stroke causes the counter element to undergo a counting motion. The counter element also includes a second region for interacting with the ratchet member. The second region includes at least one inclined surface that is engaged by the ratchet element during the return stroke of the trigger element causing the counter element to undergo a further counting motion, thereby completing a counting movement. The counter element is normally in the form of a counter ring, and is advanced partially on the outward stroke of the trigger element, and partially on the return stroke of the trigger element. As the outward stroke of the trigger can correspond to the depression of a valve stem that causes firing of the valve (and, in the case of a metered dose inhaler, also meters the contents) and the return stroke can correspond to the return of the valve stem to its resting position, this dose counter allows for precise counting of doses.

Another suitable dose counter, which is described in detail in U.S. Pat. No. 8,479,732 (Stuart et al., hereby incorporated by reference in its entirety for its disclosure of dose counters) is specially adapted for use with a metered dose inhaler. This dose counter includes a first count indicator having a first indicia bearing surface. The first count indicator is rotatable about a first axis. The dose counter also includes a second count indicator having a second indicia bearing surface. The second count indicator is rotatable about a second axis. The first and second axes are disposed such that they form an obtuse angle. The obtuse angle mentioned above can be any obtuse angle, but is advantageously 125 to 145 degrees. The obtuse angle permits the first and second indicia bearing surface to align at a common viewing area to collectively present at least a portion of a medication dosage count. One or both of the first and second indicia bearing surfaces can be marked with digits, such that when viewed together through the viewing area the numbers provide a dose count. For example, one of the first and second indicia bearing surface may have “hundreds” and “tens” place digits, and the other with “ones” place digits, such that when read together the two indicia bearing surfaces provide a number between 000 and 999 that represents the dose count.

Yet another suitable dose counter is described in U.S. Patent Application Publication No. 2012/0234317 (Stuart, hereby incorporated by reference in its entirety for its disclosure of dose counters). Such a dose counter includes a counter element that undergoes a predetermined counting motion each time a dose is dispensed. The counting motion can be vertical or essentially vertical. A count indicating element is also included. The count indicating element, which undergoes a predetermined count indicating motion each time a dose is dispensed, includes a first region that interacts with the counter element.

The counter element has regions for interacting with the count indicating element. Specifically, the counter element includes a first region that interacts with a count indicating element. The first region includes at least one surface that it engaged with at least one surface of the first region of the aforementioned count indicating element. The first region of the counter element and the first surface of the count inducing element are disposed such that the count indicating member completes a count indicating motion in coordination with the counting motion of the counter element, during and induced by the movement of the counter element, the count inducing element undergoes a rotational or essentially rotational movement. In practice, the first region of the counter element or the counter indicating element can include, for example, one or more channels. A first region of the other element can include one or more protrusions adapted to engage with said one or more channels.

Yet another dose counter is described in U.S. Pat. Nos. 8,814,035 (Stuart, hereby incorporated by reference in its entirety for its disclosure of dose counters). Such a dose counter is specially adapted for use with an inhaler with a reciprocal actuator operating along a first axis. The dose counter includes an indicator element that is rotatable about a second axis. The indicator element is adapted to undergo one or more predetermined count-indicating motions when one or more doses are dispensed. The second axis is at an obtuse angle with respect to the first axis. The dose counter also contains a worm rotatable about a worm axis. The worm is adapted to drive the indicator element. It may do this, for example, by containing a region that interacts with and enmeshes with a region of the indicator element. The worm axis and the second axis do not intersect and are not aligned in a perpendicular manner. The worm axis is also, in most cases, not disposed in coaxial alignment with the first axis. However, the first and second axes may intersect.

At least one of the various internal components of an inhaler, such as a metered dose inhaler, as described herein, such as one or more of the canister, valve, gaskets, seals, or O-rings, can be coated with one or more coatings. Some of these coatings provide a low surface energy. Such coatings are not always required because they are not always necessary for the successful operation of all inhalers. Thus, some metered dose inhalers do not include coated internal components

Some coatings that can be used are described in U.S. Pat. No. 8,414,956 (Jinks et al.), U.S. Pat. No. 8,815,325 (David et al.), and United States Patent Application Publication No. 2012/0097159 (Iyer et al.), all of which are incorporated by reference in their entireties for their disclosure of coatings for inhalers and inhaler components. Other coatings, such as fluorinated ethylene propylene resins, or FEP, are also suitable. FEP is particularly suitable for use in coating canisters.

A first acceptable coating can be provided by the following method:
a) providing one or more component of the inhaler, such as the metered dose inhaler,
b) providing a primer composition including a silane having two or more reactive silane groups separated by an organic linker group,
c) providing a coating composition including an at least partially fluorinated compound,
d) applying the primer composition to at least a portion of the surface of the component,
e) applying the coating composition to the portion of the surface of the component after application of the primer composition.

The at least partially fluorinated compound will usually include one or more reactive functional groups, with at least one reactive functional group usually being a reactive silane group, for example a hydrolysable silane group or a hydroxysilane group. Such reactive silane groups allow reaction of the partially fluorinated compound with one or more of the reactive silane groups of the primer. Often such reaction will be a condensation reaction.

One exemplary silane that can be used has the formula

$${{X}_{3 - m} ( {R}^{1} )}_{m} S i - Q - S {i ( {R}^{2} )}_{k} {X}_{3 - k}$$

wherein R¹ and R² are independently selected univalent groups, X is a hydrolysable or hydroxy group, m and k are independently 0, 1, or 2 and Q is a divalent organic linking group.

Useful examples of such silanes include one or a mixture of two or more of 1,2-bis(trialkoxysilyl)ethane, 1,6-bis(trialkoxysilyl)hexane, 1,8-bis(trialkoxysilyl)octane, 1,4-bis(trialkoxysilylethyl)benzene, bis(trialkoxysilyl)itaconate, and 4,4′-bis (trialkoxysilyl)-1,1′-diphenyl, wherein any trialkoxy group may be independently trimethoxy or triethoxy.

The coating solvent usually includes an alcohol or a hydrofluoroether.

If the coating solvent is an alcohol, preferred alcohols are C₁ to C₄ alcohols, in particular, an alcohol selected from ethanol, n-propanol, or isopropanol or a mixture of two or more of these alcohols.

If the coating solvent is an hydrofluoroether, it is preferred if the coating solvent includes a C₄ to C₁₀ hydrofluoroether. Generally, the hydrofluoroether will be of formula

$${C}_{g} {F}_{2 g + 1} O {C}_{h} {H}_{2 h + 1}$$

wherein g is 2, 3, 4, 5, or 6 and h is 1, 2, 3, or 4. Examples of suitable hydrofluoroethers include those selected from the group consisting of methyl heptafluoropropylether, ethyl heptafluoropropylether, methyl nonafluorobutylether, ethyl nonafluorobutylether and mixtures thereof.

The polyfluoropolyether silane can be of the formula

$${R}^{f} {{Q}_{v}^{1} [ {Q}_{w}^{2} - {[ {C ( {R}^{4} )}_{2} - S {i ( X )}_{3 ∼ x} {( {R}^{5} )}_{x} ]}_{y} ]}_{z}$$

wherein:
R^{ƒ} is a polyfluoropolyether moiety;
Q¹ is a trivalent linking group;
each Q² is an independently selected organic divalent or trivalent linking group;
each R⁴ is independently hydrogen or a C₁₋₄ alkyl group;
each X is independently a hydrolysable or hydroxyl group;
R⁵ is a C₁₋₈ alkyl or phenyl group;
v and w are independently 0 or 1, x is 0 or 1 or 2; y is 1 or 2; and z is 2, 3, or 4.

The polyfluoropolyether moiety R/can include perfluorinated repeating units selected from the group consisting of —(CₙF₂ₙO)—, —(CF(Z)O)—, —(CF(Z)CₙF₂ₙO)—, —(CₙF₂ₙCF(Z)O)—, —(CF₂CF(Z)O)—, and combinations thereof; wherein n is an integer from 1 to 6 and Z is a perfluoroalkyl group, an oxygen-containing perfluoroalkyl group, a perfluoroalkoxy group, or an oxygen-substituted perfluoroalkoxy group, each of which can be linear, branched, or cyclic, and have 1 to 5 carbon atoms and up to 4 oxygen atoms when oxygen-containing or oxygen-substituted, and wherein for repeating units including Z the number of carbon atoms in sequence is at most 6. In particular, n can be an integer from 1 to 4, more particularly from 1 to 3. For repeating units including Z the number of carbon atoms in sequence may be at most four, more particularly at most 3. Usually, n is 1 or 2 and Z is a —CF₃ group, more wherein z is 2, and R^{ƒ} is selected from the group consisting of —CF₂O(CF₂O)ₘ(C₂F₄O)ₚCF₂—, —CF(CF₃)O(CF(CF₃)CF₂O)ₚCF(CF₃)—, —CF₂O(C₂F₄O)ₚCF₂—, —(CF₂)₃O(C₄F₈O)ₚ(CF₂)₃—, —CF(CF₃)—(OCF₂CF(CF₃))ₚO—CₜF₂ₜ—O(CF(CF₃)CF₂O)ₚCF(CF₃)—, wherein t is 2, 3 or 4 and wherein m is 1 to 50, and p is 3 to 40.

A cross-linking agent can be included. Exemplary cross-linking agents include tetramethoxysilane; tetraethoxysilane; tetrapropoxysilane; tetrabutoxysilane; methyl triethoxysilane; dimethyldiethoxysilane; octadecyltriethoxysilane; 3-glycidoxy-propyltrimethoxysilane; 3-glycidoxy-propyltriethoxysilane; 3-aminopropyl-trimethoxysilane; 3-aminopropyl-triethoxysilane; bis(3-trimethoxysilylpropyl) amine; 3-aminopropyl tri (methoxyethoxyethoxy) silane; N-(2-aminoethyl)3-aminopropyltrimethoxysilane; bis(3-trimethoxysilylpropyl) ethylenediamine; 3-mercaptopropyltrimethoxysilane; 3-mercaptopropyltriethoxysilane; 3-trimethoxysilyl-propylmethacrylate; 3-triethoxysilypropylmethacrylate; bis (trimethoxysilyl) itaconate; allyltriethoxysilane; allyltrimethoxysilane; 3-(N-allylamino) propyltrimethoxysilane; vinyltrimethoxysilane; vinyltriethoxysilane; and mixtures thereof.

The component to be coated can be pre-treated before coating, such as by cleaning. Cleaning can be by way of a solvent, such as a hydrofluoroether, e.g., HFE-72DE, or an azeotropic mixture of 70% w/w (i.e., weight percent) trans-dichloroethylene; 30% w/w of a mixture of methyl and ethyl nonafluorobutyl and nonafluoroisobutyl ethers.

The above-described first acceptable coating is particularly useful for coating valves components, including one or more of valve stems, bottle emptiers, springs, and tanks. This coating system can be used with any type of inhaler and any formulation described herein.

In some embodiments the actuator nozzle is sized so as to optimize the fine particle fraction (FPF) and/or respirable dose delivered of the formulation within the canister. In some embodiments the cross-sectional shape of the actuator nozzle is essentially circular or circular and has a predetermined diameter. In some embodiments where the cross-sectional shape of the actuator nozzle is non-circular, for example oval, an effective diameter may be determined by taking an average over the distances spanning the opening (e.g., the average of major and minor axes of an ellipse).

In some embodiments the exit orifice (effective diameter) of the actuator nozzle may be 0.08 mm or greater, 0.10 mm or greater, 0.12 mm or greater, 0.15 mm or greater, 0.175 mm or greater, 0.225 mm or greater 0.3 mm or greater, or 0.4 mm or greater. In some embodiments the exit orifice (effective diameter) of the actuator nozzle may be 0.5 mm or less, 0.4 mm or less, 0.3 mm or less, 0.225 mm or less, 0.175 mm or less, or 0.15 mm or less. In some embodiments the exit orifice (effective diameter) of the actuator nozzle may be 0.12 mm to 0.5 mm, 0.12 mm to 0.4 mm, 0.12 mm to 0.3 mm, 0.12 mm to 0.225 mm, 0.12 mm to 0.175 mm, or 0.12 mm to 0.15 mm. In some embodiments the exit orifice (effective diameter) of the actuator nozzle may be 0.15 mm to 0.5 mm, 0.15 mm to 0.4 mm, 0.15 mm to 0.3 mm, 0.15 mm to 0.225 mm, or 0.15 mm to 0.175 mm. In some embodiments the exit orifice (effective diameter) of the actuator nozzle may be 0.175 mm to 0.5 mm, 0.175 mm to 0.4 mm, 0.175 mm to 0.3 mm, or 0.175 mm to 0.225 mm. In some embodiments the exit orifice (effective diameter) of the actuator nozzle may be 0.12 mm to 0.5 mm, 0.14 mm to 0.4 mm, or 0.18 mm to 0.3 mm. In some embodiments the exit orifice (effective diameter) of the actuator nozzle may be 0.12 mm to 0.3 mm or 0.18 mm to 0.22 mm. In some embodiments the exit orifice (effective diameter) of the actuator nozzle may be 0.12 mm to 0.25 mm.

It should be appreciated by one of ordinary skill in the art that a given actuator nozzle exit orifice may not be suitable for delivery of any formulation, and that selection of a suitable actuator nozzle exit orifice for a given formulation involves considerable effort.

In some embodiments, the MDI is manufactured by pressure filling. In pressure filling, the liquid or powdered medicament, combined with one or more excipients (e.g., co-solvents), is placed in a suitable aerosol container (i.e., canister) capable of withstanding the vapor pressure of the propellant and fitted with a metering valve prior to filling. The propellant is then forced as a liquid through the valve into the container. In an alternate process of pressure filling, the particulate drug is combined in a process vessel with propellant and one or more excipients (e.g., cosolvents), and the resulting drug solution is transferred through the metering valve fitted to a suitable MDI container.

In some embodiments, the MDI is manufactured by cold filling. In cold filling, the liquid or powdered medicament is combined with one or more excipients (e.g., co-solvents) and propellant which is chilled below its boiling point and, optionally, one or more excipients are added to the MDI container. In addition, a metering valve is fitted to the container post filling.

For both pressure filling and cold filling processes, additional steps, such as mixing, sonication, and homogenization may be optionally employed.

### Embodiments

Embodiment 1 is a metered dose inhaler comprising: a metering valve; a canister; and an actuator comprising an actuator nozzle; wherein the canister comprises a formulation, the formulation comprising greater than 70% by weight of propellant HFO-1234ze(E), and at least one active pharmaceutical ingredient dissolved in the formulation to form a solution.

Embodiment 2 is the inhaler of embodiment 1, wherein the active pharmaceutical ingredient is selected from beta agonists (short- or long-acting beta agonists), corticosteroids, anticholinergic agents, TYK inhibitors, and combinations thereof.

Embodiment 3 is the inhaler of embodiment 1, wherein the active pharmaceutical ingredient comprises a corticosteroid.

Embodiment 4 is the inhaler of embodiment 2 or 3, wherein the corticosteroid is selected from beclomethasone and ciclesonide.

Embodiment 5 is the inhaler of embodiment 1, wherein the active pharmaceutical ingredient comprises an anticholinergic agent.

Embodiment 6 is the inhaler of embodiment 2 or 5, wherein the anticholinergic agent is selected from ipratropium, tiotropium, aclidinium, umeclidinium, and glycopyrronium (i.e., glycopyrrolate).

Embodiment 7 is the inhaler of embodiment 1, wherein the active pharmaceutical ingredient comprises a beta agonist (short- or long-acting).

Embodiment 8 is the inhaler of embodiment 2 or 7, wherein the beta agonist (short or long-acting beta agonist) is selected from formoterol, indacaterol, olodaterol, vilanterol, and abediterol.

Embodiment 9 is the inhaler of embodiment 1, wherein the active pharmaceutical ingredient comprises a TYK inhibitor (e.g., nintedanib).

Embodiment 10 is the inhaler of any preceding embodiment, wherein the formulation comprises at least two (in some embodiments, two or three, and in some embodiments, two) active pharmaceutical agents in solution.

Embodiment 11 is the inhaler of embodiment 10, wherein one active pharmaceutical ingredient is a beta agonist (short- or long-acting) and one active pharmaceutical ingredient is a corticosteroid.

Embodiment 12 is the inhaler of embodiment 11, wherein the formulation further comprises an anticholinergic agent in solution.

Embodiment 13 is a metered dose inhaler comprising: a metering valve; a canister; and an actuator comprising an actuator nozzle; wherein the canister comprises a formulation, the formulation comprising a propellant comprising HFO-1234ze(E), and an active pharmaceutical ingredient comprising beclomethasone or a pharmaceutically acceptable salt or ester thereof, wherein the beclomethasone or pharmaceutically acceptable salt or ester thereof is dissolved in the formulation to form a solution.

Embodiment 14 is the inhaler of embodiment 13, wherein the beclomethasone or a pharmaceutically acceptable salt or ester thereof is the sole pharmaceutical ingredient.

Embodiment 15 is the inhaler of embodiment 13 or 14, wherein the beclomethasone or a pharmaceutically acceptable salt or ester thereof is beclomethasone dipropionate.

Embodiment 16 is a metered dose inhaler comprising: a metering valve; a canister; and an actuator comprising an actuator nozzle; wherein the canister comprises a formulation, the formulation comprising a propellant comprising HFO-1234ze(E), and an active pharmaceutical ingredient comprising ciclesonide or a pharmaceutically acceptable salt or ester thereof, wherein the ciclesonide or pharmaceutically acceptable salt or ester thereof is dissolved in the formulation to form a solution.

Embodiment 17 is a metered dose inhaler comprising: a metering valve; a canister; and an actuator comprising an actuator nozzle; wherein the canister comprises a formulation, the formulation comprising a propellant comprising HFO-1234ze(E), and an active pharmaceutical ingredient comprising formoterol or a pharmaceutically acceptable salt or ester thereof, wherein the formoterol or pharmaceutically acceptable salt or ester thereof is dissolved in the formulation to form a solution.

Embodiment 18 is the inhaler of embodiment 17, wherein the formoterol or a pharmaceutically acceptable salt or ester thereof is the sole active pharmaceutical ingredient.

Embodiment 19 is the inhaler of embodiment 17 or 18, wherein the formoterol or a pharmaceutically acceptable salt or ester thereof is formoterol fumarate.

Embodiment 20 is a metered dose inhaler comprising: a metering valve; a canister; and an actuator comprising an actuator nozzle; wherein the canister comprises a formulation, the formulation comprising: a propellant comprising HFO-1234ze(E), and an active pharmaceutical ingredient comprising formoterol or a pharmaceutically acceptable salt or ester thereof and beclomethasone or a pharmaceutically acceptable salt or ester thereof, wherein the formoterol or pharmaceutically acceptable salt or ester thereof and the beclomethasone or a pharmaceutically acceptable salt or ester thereof are dissolved in the formulation to form a solution.

Embodiment 21 is the inhaler of embodiment 20, wherein the formoterol or a pharmaceutically acceptable salt or ester thereof is formoterol fumarate and the beclomethasone or a pharmaceutically acceptable salt or ester thereof is beclomethasone dipropionate.

Embodiment 22 is a metered dose inhaler comprising: a metering valve; a canister; and an actuator comprising an actuator nozzle; wherein the canister comprises a formulation, the formulation comprising a propellant comprising HFO-1234ze(E), and an active pharmaceutical ingredient comprising tiotropium or a pharmaceutically acceptable salt or ester thereof, wherein the tiotropium or pharmaceutically acceptable salt or ester thereof is dissolved in the formulation to form a solution.

Embodiment 23 is the inhaler of embodiment 22, wherein the tiotropium or a pharmaceutically acceptable salt or ester thereof is the sole active pharmaceutical ingredient.

Embodiment 24 is the inhaler of embodiment 22 or 23, wherein the tiotropium or a pharmaceutically acceptable salt or ester thereof is tiotropium bromide.

Embodiment 25 is the inhaler of any preceding embodiment, wherein HFO-1234ze(E) is the sole propellant.

Embodiment 26 is the inhaler of any of embodiments 1 to 24, wherein propellant comprises HFO-1234ze(E) and another hydrofluroroolefin or a hydrofluoroalkane.

Embodiment 27 is the inhaler of embodiment 26, wherein the formulation includes the other hydrofluroroolefin or hydrofluoroalkane in an amount of 0.1% to 20% by weight, of the total formulation.

Embodiment 28 is the inhaler of embodiment 27, wherein the formulation includes the other hydrofluroroolefin or hydrofluoroalkane in an amount of 0.1% to 5% by weight, of the total formulation.

Embodiment 29 is the inhaler of embodiment 28, wherein the formulation includes the other hydrofluroroolefin or hydrofluoroalkane in an amount of 0.1% to 0.5% by weight, of the total formulation.

Embodiment 30 is the inhaler of any preceding embodiment further comprising polyethylene glycol (e.g., PEG 300 or PEG 1000) or water.

Embodiment 31 is the inhaler of embodiment 30, wherein the formulation includes the polyethylene glycol or water in an amount of 0.01% to 1% by weight (based on the total weight of the formulation).

Embodiment 32 is the inhaler of any preceding embodiment, wherein the formulation includes the API at a concentration of from 0.0008% to 6.8% by weight (or 0.01% to 1.0% by weight, or 0.05% to 0.5% by weight) of the total composition.

Embodiment 33 is the inhaler of any preceding embodiment, wherein the formulation further comprises ethanol.

Embodiment 34 is the inhaler of embodiment 33, wherein the amount of ethanol by weight of the total formulation is between 0.2% and 20%.

Embodiment 35 is the inhaler of embodiment 34, wherein the amount of ethanol by weight of the total formulation is between 0.5% and 20%.

Embodiment 36 is the inhaler of embodiment 35, wherein the amount of ethanol by weight of the total formulation is between 2% and 20%.

Embodiment 37 is the inhaler of embodiment 36, wherein the amount of ethanol by weight of the total formulation is between 2% and 10%.

Embodiment 38 is the inhaler of embodiment 36, wherein the amount of ethanol by weight of the total formulation is between 15% and 20%.

Embodiment 39 is the inhaler of any preceding embodiment, wherein the formulation further comprises an organic acid, inorganic acid, or a combination thereof.

Embodiment 40 is the inhaler of embodiment 39, wherein the acid is an inorganic acid.

Embodiment 41 is the inhaler of embodiment 40, wherein the inorganic acid is selected from hydrochloric acid, nitric acid, phosphoric acid, sulfuric acid, and a combination thereof.

Embodiment 42 is the inhaler of embodiment 41, wherein the acid is hydrochloric acid.

Embodiment 43 is the inhaler of embodiment 39, wherein the acid is an organic acid.

Embodiment 44 is the inhaler of embodiment 43, wherein the organic acid is selected from citric acid, ascorbic acid, maleic acid, acetic acid, succinic acid, formic acid, and a combination thereof.

Embodiment 45 is the inhaler of embodiment 44, wherein the acid is citric acid.

Embodiment 46 is the inhaler of any of embodiments 39 to 45, wherein the amount of acid by weight of the total formulation is between 0.001% and 1.0%.

Embodiment 47 is the inhaler of embodiment 46, wherein the amount of acid by weight of the total formulation is between 0.004% and 0.4%.

Embodiment 48 is the inhaler of embodiment 47, wherein the amount of acid by weight of the total formulation is between 0.04% and 0.4%.

Embodiment 49 is the inhaler of any preceding embodiment, wherein the metering valve comprises a metering chamber having a size between 25 microliters and 200microliters.

Embodiment 50 is the inhaler of embodiment 49, wherein the metering chamber of the metering valve has a size between 25 microliters and 100 microliters (or between 50 microliters and 100 microliters, between 50 microliters and 65 microliters, or between 50 microliters and 63 microliters).

Embodiment 51 is the inhaler of any of embodiments 13 to 50, wherein the formulation comprises greater than 70% by weight of propellant HFO-1234ze (E), based on the total weight of the formulation.

Embodiment 52 is the inhaler of any preceding embodiment, wherein the formulation comprises at least 80%, greater than 80%, at least 85%, greater than 85%, at least 90%, or greater than 90%, by weight of propellant HFO-1234ze (E), based on the total weight of the formulation.

Embodiment 53 is the inhaler of any preceding embodiment, wherein the amount of HFO-1234ze(E) by weight of the total propellant in the formulation is greater than 95%.

Embodiment 54 is the inhaler of embodiment 53, wherein the amount of HFO-1234ze(E) by weight of the total propellant in the formulation is greater than 99%.

Embodiment 55 is the inhaler of any preceding embodiment, wherein the actuator exit orifice diameter is 0.12 mm to 0.5 mm.

Embodiment 56 is the inhaler of embodiment 56, wherein the actuator exit orifice diameter is 0.15 mm to 0.3 mm.

Embodiment 57 is the inhaler of embodiment 56, wherein the actuator exit orifice diameter is 0.175 mm to 0.225 mm.

Embodiment 58 is the inhaler of any preceding embodiment, wherein the amount of formulation in the canister is 1 mL to 30 mL.

Embodiment 59 is the inhaler of any preceding embodiment, wherein the canister contains a predetermined number of doses that is from 30 to 200.

Embodiment 60 is the inhaler of any preceding embodiment, which does not include coated internal components.

Embodiment 61 is the inhaler of any preceding embodiment, wherein the actuator nozzle comprises an exit orifice effective diameter of 0.12 mm to 0.3 mm.

### EXAMPLES

### Comparative Example 1

### Solubility of Tiotropium Bromide in HFA-134a and HFO-1234ze(E)

In this example, saturated solutions of tiotropium bromide (TB) were prepared in two different propellants by adding an excess of drug to ensure saturated solubility was achieved. A first saturated solution including TB and the propellant HFO-1234ze(E) (1,3,3,3-tetrafluoropropene) was prepared. A second saturated solution including TB and the propellant HFA-134a (1,1,1,2-tetrafluoroethane) was prepared. For both saturated solution compositions, the concentration of tiotropium bromide monohydrate (TBM) added was 0.3750 mg/mL. The saturated solutions did not contain any other added components. In both cases an excess of non-dissolved drug was present after addition of TBM at this concentration

A second set of saturated solutions was prepared using each propellant with the addition of 20% ethanol by weight and 0.4% citric acid by weight. The TB solubility of each saturated solution was measured by content assay following filtration of the excess non-dissolved drug. It was observed that TB was practically insoluble in both HFO-1234ze(E) and HFA-134a. Data from this example is shown in Table 1.

TABLE 1

| Solution composition | Mean TB solubility (mg/mL) |
|---|---|
| HFO-1234ze(E) | Below quantification |
| HFO-1234ze(E), 20% ethanol, | 0.0331 |
| 0.4% citric acid | |
| HFA-134a | Below quantification |
| HFA-134a, 20% ethanol, 0.4% | 0.1220 |
| citric acid | |

From this example, it was learned that TB was practically insoluble in compositions including only TB and propellant. It was also learned that TB was partially soluble in compositions of each propellant with 20% ethanol and 0.4% citric acid.

### Example 2

### Solubility and Physical Stability of Tiotropium Bromide in Compositions Including HFO-1234ze(E), Ethanol, and Different Acids

In this example, solutions of TB in HFO-1234ze(E) including different amounts of ethanol and different acids were tested.

Solutions were prepared of 0.1204 mg/mL TBM in HFO-1234ze (E) with 10%, 12.5%, 15%, 17.5%, 20%, or 22.5% by weight of ethanol. An acid was added to each solution to test the interaction between TB, the given weight percentage of ethanol, and the acid. Six acids in total were tested in solutions across a different range of ethanol and acid concentration levels. Acids tested included citric acid, acetic acid, hydrochloric acid (HCl), succinic acid, ascorbic acid, and sulfuric acid. Solubility and physical stability of TB in each solution was visually inspected for up to 21 days at room temperature.

It was observed with all organic acids between 0.004% and 0.4%, and all inorganic acids, between 0.004% and 0.04% caused TB to precipitate from solution, with the exception of citric acid,. Solutions of 0.1250 mg/mL TB with citric acid concentrations between 0.04% and 0.4% by weight citric acid and each of the six tested weight percentages of ethanol were prepared and visually analyzed for seven days. It was observed that in compositions including less than 17.5% ethanol and each concentration of acid, TB precipitated from solution.

From this example, it was learned that formulations of TB in HFO-1234ze (E) with at least 17.5% by weight ethanol and with greater than 0.04% by weight citric acid were visually stable and remained in solution up to seven days post-preparation.

### Example 3

### Chemical Stability of Tiotropium Bromide in Compositions Including in HFO-1234ze(E), Ethanol, and Citric Acid

Three solutions including TB, ethanol, citric acid, and the propellant HFO-1234ze(E) were prepared. For all three solutions, the concentration of ethanol was 20% by weight and the concentration of TBM was 0.1250 mg/mL. The first solution included 0.04% by weight citric acid. The second solution included 0.22% by weight citric acid. The third solution included 0.4% by weight citric acid. Each solution was pressure filled into an FEP-coated canister and fitted with a 50-μL BESPAK valve. The filled canisters were stored at 40° C. and 75% relative humidity for two weeks to simulate aging. Three replicates of each solution in total were prepared, packed, and stored.

After initial preparation and following two weeks' storage, each filled canister was analyzed for TB content and presence of impurities/degradants. Each solution was observed to have only a slight decrease in TB content after two weeks storage relative to the initial measured TB content as shown in Table 2. Each solution was also observed to have a low level of total impurities and known tiotropium degradants (with all impurities/degradants less than 0.2% by weight) as shown in Table 3.

TABLE 2

| | Citric Acid Level (% w/w) | | |
|---|---|---|---|
| Timepoint | 0.04% | 0.22% | 0.4% |
| Initial | 0.1026 | 0.1026 | 0.1012 |
| 2 weeks | 0.1009 | 0.1011 | 0.1001 |

TABLE 3

| | | Citric Acid Level (% w/w) | | |
|---|---|---|---|---|
| Timepoint | Impurity | 0.04% | 0.22% | 0.4% |
| Initial | Unknown 22 | 0.05 | 0.04 | 0.06 |
| | Unknown 21 | — | 0.04 | 0.03 |
| | Unknown 11 | 0.05 | 0.04 | 0.03 |
| | Total UV impurities | 0.10 | 0.08 | 0.13 |
| 2 weeks | Ph. Eur. Impurity B | 0.11 | 0.08 | 0.08 |
| | Ethyl Dithienyl Glycolate | 0.05 | 0.04 | 0.06 |
| | Tiotropium Ethyl Ether | — | — | 0.05 |
| | Total UV impurities | 0.16 | 0.12 | 0.19 |

From this example, it was learned that compositions of TB in HFO-1234ze(E), 20% ethanol and citric acid at levels between 0.04% to 0.4% citric acid were relatively chemically stable over storage for two weeks at 40° C. and 75% relative humidity.

### Example 4

### Comparison of Actuator Exit Orifice Sizes for Delivery of Tiotropium Bromide in Compositions of HFO-1234ze(E), Ethanol, and Citric Acid

A solution composition including 0.125 mg/mL TBM, 0.22% by weight citric acid, and 20% by weight ethanol in HFO-1234ze(E) was prepared. The solution was pressure filled into FEP-coated canisters fitted with a 50-μL BESPAK Valve. Three units were prepared in total. One unit was tested with a KINDEVA DRUG DELIVERY (KDD) actuator with a 0.3-mm exit orifice, one unit was tested with a KDD actuator with a 0.22-mm exit orifice, and one unit was tested with a KDD actuator with a 0.18-mm exit orifice

The fine particle mass (FPM) smaller than 5 μm per actuation, median mass aerodynamic diameter (MMAD), delivered dose (ex-act), and geometric standard deviation (GSD) were measured for each unit tested. These measurements are shown in Table 4.

TABLE 4

| Exit orifice | FPM | MMAD | Total Ex-Act | |
|---|---|---|---|---|
| (mm) | (μg/act) | (μm) | (μg) | GSD |
| 0.18 | 2.4573 | 1.6537 | 4.6321 | 1.8323 |
| 0.22 | 1.5875 | 1.4980 | 4.7161 | 1.8477 |
| 0.30 | 0.9230 | 1.3393 | 4.5745 | 1.8817 |

From this example, it was observed that FPM is increased when the formulation was delivered using progressively smaller actuator exit orifices. It was learned that actuator exit orifices between 0.18 mm and 0.22 mm were preferred for delivery of anticipated appropriate FPM of a solution of TBR in HFO-1234ze(E) with 20% ethanol by weight and 0.04% to 0.4% citric acid by weight.

### Example 5

### Solutions of Beclomethasone Dipropionate in HFO-1234ze(E) With Ethanol

Two different solutions of beclomethasone dipropionate (BDP) and ethanol were prepared in HFO-1234ze(E). A first solution included BDP at concentration of 1 mg/mL. A second solution included BDP at a concentration of 2 mg/mL. Both solutions included a concentration of ethanol of 8.0% by weight. The amount of BDP (1 mg/mL and 2 mg/mL) in each solution was selected to provide a nominal dose of 50 μg/actuation and 100 μg/actuation, respectively, from a 50-μL valve. The solutions were cold filled into uncoated aluminum and FEP-coated aluminum canisters.

For the 1 mg/mL solution, delivered dose with an actuator having an actuator exit orifice diameter of 0.3 mm was 42.3 μg/actuation. Fine particle mass was 27 μg/actuation.

For the 2 mg/mL solution, delivered dose with an actuator having an actuator exit orifice diameter of 0.3 mm was 80.3 μg/actuation. Fine particle mass was 54 μg/actuation.

From this example, it was learned that solutions of 1 mg/mL and 2 mg/mL BDP and 8% ethanol in HFO-1234ze(E) resulted in a measured delivered dose and fine particle dose consistent with the desired appropriate doses.

### Example 6

### Solutions of Formoterol Fumarate in HFO-1234ze(E)

A solution of formoterol fumarate (FF), HCl, and ethanol was prepared in HFO-1234ze(E). The concentration of 1 molar (M) HCl was 0.024% by weight. The concentration of ethanol was 12.0% by weight. The amount of FF (0.114 mg/mL) was selected to provide a nominal actuation of dose of 6 μg/actuation from a 50-μL valve. The formulation was pressure filled or cold filled into polyethylene terephthalate (PET) vials to allow for visual observation. All components visually observed to be soluble and physically stable both initially and on observation for up to 5 weeks when stored at ambient conditions. As formulations of FF would typically be refrigerated during storage, this ambient storage period simulated accelerated aging.

From this example, it was learned that 0.114 mg/mL FF was soluble and physically stable in a solution of 12.0% ethanol, and 0.024% HCl and HFO-1234ze(E) when cold filled or pressure filled for up to five weeks at ambient conditions.

### Example 7

### Solutions Including Beclomethasone Dipropionate and Formoterol Fumarate in HFO-1234ze(E).

In this example, a solution including BDP and FF was prepared and tested. Solubility and physical stability of the solution was determined by visual assessment over five weeks. Pharmaceutical product performance metrics were assayed to demonstrate uniformity of delivered dose though unit life and the influence of actuator exit orifice diameters on fine particle fraction.

A solution of BDP, FF, HCl, and ethanol was prepared in HFO-1234ze(E). The concentration of 1M hydrochloric acid was 0.024% by weight. The concentration of ethanol was 12% by weight. The amount of BDP (2 mg/mL) was selected to provide a nominal dose of 100 μg/actuation from a 50-μL valve. The amount of FF (0.114 mg/mL) was selected to provide a nominal dose of 6 μg/actuation from a 50-μL valve.

The formulation for visual assessment was pressure filled or cold filled into PET vials. All components were soluble, and the resulting solution was physically stable both initially and on observation for up to 5 weeks at ambient conditions.

The formulation for pharmaceutical product performance testing was cold filled into FEP-coated aluminum canisters. Each canister was fitted with a 50-μL BESPAK valve For uniformity of delivered dose testing, a KDD actuator with a 0.3-mm exit orifice diameter was used. For Fine Particle Fraction (FPF) testing, KDD actuators with a range of exit orifice diameters as described in Table 3 were used.

Content assay of the prepared units was within +/−10% of target for both BDP and FF. Uniformity of delivered dose testing (ex-actuator) showed no trending between start, middle, and end of unit life. FPF across a range of actuator exit orifice diameters was measured using a Fast-Screening Impactor (FSI) coupled to an OPC EMMACE anatomical throat model. The FPF results for BDP and FF are shown in Table 5 below.

TABLE 5

| Actuator Exit Orifice Diameter (mm) | FF FPF (%) | BDP FPF (%) |
|---|---|---|
| 0.12 | 76.5 | 76.2 |
| 0.15 | 73.2 | 72.8 |
| 0.18 | 68.7 | 68.8 |
| 0.22 | 58.3 | 58.5 |
| 0.30 | 44.7 | 45.1 |

From this example, it was learned that all components in a solution of 2 mg/ml BDP, 0.114 mg/mL FF, 12% ethanol, 0.024% HCl and HFO-1234ze(E) were soluble, and that the resulting solution was physically stable both initially and on observation for up to 5 weeks at ambient conditions when cold filled or pressure filled into PET vials.

It was also learned that this solution exhibited consistent uniformity of delivered dose through unit life. It was also learned that the particle size of the solution aerosol could be manipulated using actuators having different exit orifice diameters to achieve a wide range of fine particle fractions and hence fine particle doses for both BDP and FF.

### Example 8

### Stability of Solutions of Beclomethasone Dipropionate and Formoterol Fumarate in HFO-1234ze(E)

Low-strength and high-strength solutions of BDP, FF, HCl, and ethanol were prepared in HFO-1234ze(E). The concentration of ethanol was 12.0% by weight in both solutions.

Low-strength solution. The amount of BDP was 2 mg/mL and the amount of FF was 0.114 mg/mL. The concentration of 1M HCl was 0.024% by weight.

High-strength solution. The amount of BDP was 3.1746 mg/mL and the amount of FF was 0.0903 mg/mL. The concentration of 1M HCl was 0.019% by weight.

The balance of the composition was HFO-1234ze(E) for both solutions.

The canisters used were 10 mL plain aluminum canisters, fitted with either a 50-μL BESPAK valve on the low strength solution or a 63-μL BESPAK valve on the high strength solution, and KDD actuators with 0.3-mm exit orifice diameter and 0.65-mm jet length, with a dose counter. The fill weight of the canisters, to provide 120 actuations, was 10.2 g (+/−0.2 g) for the low strength solution and 12.1 g (+/−0.2 g) for the high strength solution,

A concentrate of BDP, FF, HCl, and ethanol was prepared in a glass jar with sonication until a visually clear solution was obtained. Concentrate was then added to open individual canisters at the required amount. BESPAK valves were placed on the filled canisters and crimped (without vacuum purge). HFO-1234ze(E) propellant was then pressure filled through the valve to achieve the overall target unit fill weight.

Units of both solutions were placed valve down in a 25° C./60% relative humidity stability cabinet for testing at 6 or 13 weeks. Matching placebo units were manufactured in the same way and placed in the stability cabinet.

The results for mean delivered dose, mean fine particle mass, and mean total impurities for BDP and FF in both solutions are shown in Table 6.

TABLE 6

| | Low Strength | | | High Strength | | |
|---|---|---|---|---|---|---|
| | | T = | T = | | T = | T = |
| | Initial | 6 wk | 13 wk | Initial | 6 wk | 13 wk |
| BDP Mean | 83.6 | 86.1 | 85.5 | 189.9 | 187.3 | 192.4 |
| Delivered Dose | | | | | | |
| (μg/act) | | | | | | |
| FF Mean | 5.0 | 4.8 | 4.7 | 5.1 | 4.7 | 4.9 |
| Delivered Dose | | | | | | |
| (μg/act) | | | | | | |
| BDP Mean | 45.2 | 47.7 | 47.5 | 88.2 | 99.2 | 99.9 |
| Fine Particle | | | | | | |
| Mass* | | | | | | |
| (μg/act) | | | | | | |
| FF Mean | 2.7 | 2.6 | 2.6 | 2.4 | 2.5 | 2.5 |
| Fine Particle | | | | | | |
| Mass* | | | | | | |
| (μg/act) | | | | | | |
| BDP Mean | 0.13 | 0.06 | 0.22 | 0.15 | 0.07 | 0.21 |
| Total | | | | | | |
| Impurities | | | | | | |
| (% w/w) | | | | | | |
| FF Mean | 0.51 | 1.83 | 2.61 | 0.22 | 1.09 | 2.17 |
| Total | | | | | | |
| Impurities | | | | | | |
| (% w/w) | | | | | | |

*<5 μm

From this example, it was learned that both BDP/FF solution product strengths in HFO-1234ze(E) are physically stable, as demonstrated by their consistent pharmaceutical product performance (measured delivered dose and fine particle mass/dose consistent with the desired appropriate doses) and chemically stable as demonstrated by low level of impurities profiles over the 13-week accelerated aging period

Both BDP/FF solution product strengths in HFO1234ze (E) also resulted in a measured delivered dose and fine particle mass/dose consistent with the desired pMDI appropriate doses.

The embodiments described above and illustrated in the figures are presented by way of example only and are not intended as a limitation upon the concepts and principles of the present disclosure. As such, it will be appreciated by one having ordinary skill in the art that various changes in the elements and their configuration and arrangement are possible without departing from the spirit and scope of the present disclosure. All references and publications cited herein are expressly incorporated herein by reference in their entirety into this disclosure. Various features and aspects of the present disclosure are set forth in the following claims.

1. A metered dose inhaler comprising:
a metering valve;
a canister; and
an actuator comprising an actuator nozzle;
wherein the canister comprises a formulation, the formulation comprising greater than 70% by weight of propellant HFO-1234ze(E), and at least one active pharmaceutical ingredient dissolved in the formulation to form a solution.

2. The inhaler of claim 1, wherein the active pharmaceutical ingredient is selected from beta agonists, corticosteroids, anticholinergic agents, TYK inhibitors, and combinations thereof.

3. The inhaler of claim 2, wherein the corticosteroid is selected from beclomethasone and ciclesonide.

4. The inhaler of claim 2, wherein the anticholinergic agent is selected from ipratropium, tiotropium, aclidinium, umeclidinium, and glycopyrronium.

5. The inhaler of claim 2, wherein the beta agonist is selected from formoterol, indacaterol, olodaterol, vilanterol, and abediterol.

6. The inhaler of claim 1, wherein the formulation comprises at least two active pharmaceutical ingredients in solution.

7. The inhaler of claim 6, wherein one active pharmaceutical ingredient is a short- or long-acting beta agonist and one active pharmaceutical ingredient is a corticosteroid in solution.

8. The inhaler of claim 7, wherein the formulation further comprises an anticholinergic agent in solution.

9. A metered dose inhaler comprising:
a metering valve;
a canister; and
an actuator comprising an actuator nozzle;
wherein the canister comprises a formulation, the formulation comprising a propellant comprising HFO-1234ze(E), and an active pharmaceutical ingredient comprising:
beclomethasone or a pharmaceutically acceptable salt or ester thereof, wherein the beclomethasone or pharmaceutically acceptable salt or ester thereof is dissolved in the formulation to form a solution; and/or
formoterol or a pharmaceutically acceptable salt or ester thereof, wherein the formoterol or pharmaceutically acceptable salt or ester thereof is dissolved in the formulation to form a solution.

10. (canceled)

11. (canceled)

12. A metered dose inhaler comprising:
a metering valve;
a canister; and
an actuator comprising an actuator nozzle;
wherein the canister comprises a formulation, the formulation comprising a propellant comprising HFO-1234ze(E), and an active pharmaceutical ingredient comprising tiotropium or a pharmaceutically acceptable salt or ester thereof, wherein the tiotropium or pharmaceutically acceptable salt or ester thereof is dissolved in the formulation to form a solution.

13. The inhaler of claim 1, wherein HFO-1234ze(E) is the sole propellant.

14. The inhaler of claim 1, wherein the formulation further comprises ethanol.

15. The inhaler of claim 14, wherein the amount of ethanol by weight of the total formulation is between 5% and 20%.

16. The inhaler of claim 1, wherein the formulation further comprises an organic acid, an inorganic acid, or a combination thereof.

17. The inhaler of claim 16, wherein the formulation comprises 0.04% to 0.4% acid by weight of the total formulation.

18. The inhaler of claim 1, wherein the metering valve comprises a metering chamber having a size between 25 microliters and 200 microliters.

19. The inhaler of claim 1, which does not comprise coated internal components.

20. The inhaler of claim 1, wherein the actuator nozzle comprises an exit orifice effective diameter of 0.12 mm to 0.3 mm.

21. The inhaler of claim 16, wherein the formulation comprises citric acid.

22. The inhaler of claim 9, wherein the amount of HFO-1234ze(E) by weight of the total propellant in the formulation is at least 90%.
